(19) United States
(12) Patent Application Publication
(10) Pub. No.: US 2026/0232721 A1
(43) Pub. Date: Aug. 13, 2026

(54) PRESERVATIVE-FREE ALLERGEN BLOCKER EYE GEL

(71) Applicants: PROCYTO LABS PVT LTD, Bhubaneswar (IN); HYDERABAD EYE RESEARCH FOUNDATION, Hyderabad (IN)

(72) Inventors: Andrea LEONARDI, Padova (IT); Srikant K Sahu, Bhubaneswar (IN); Neera Singh, Bhubaneswar (IN); Anjani Kumar UPADHYAY, Bhubaneswar (IN); Mousumi Meghamala NAYAK

(73) Assignees: PROCYTO LABS PVT LTD, Bhubaneswar (IN); HYDERABAD EYE RESEARCH FOUNDATION, Hyderabad (IN)

(21) Appl. No.: 19/470,868

(22) PCT Filed: Mar. 30, 2023
(86) PCT No.: PCT/IB2024/053126

§ 371 Date: Sep. 30, 2025

(30) Foreign Application Priority Data
Mar. 30, 2023 (IN) 202331024459

(51) Int. Cl. A61K 31/728; A61K 9/06; A61K 31/715; A61K 31/717; A61K 36/48; A61K 47/10; A61P 27/02; A61P 37/08

(52) U.S. Cl. CPC A61K 31/728; A61K 9/06; A61K 31/715; A61K 31/717; A61K 36/48; A61K 47/10; A61P 27/02; A61P 37/08; A61K 2236/331; A61K 2236/53

(57) ABSTRACT

The present disclosure relates to a preservative-free allergen blocker eye gel. More particularly, it deals with a novel in situ gel formulation designed to prevent chronic eye allergies. Moreover, the said formulation converts into a gel upon contacting with tears, forming a protective layer over the ocular surface. In addition, the said formulation is preservative-free with a proven safety profile, it does not comprise of any drug. In a preferred embodiment, the formulation is compatible with ocular tissues, not impairing visual light transmission or causing irritation. The said formulation block the entry of allergens. Furthermore, the formulation can be used to block pollutants, prevent moisture loss, and restore epithelial integrity.

This application claims priority from PCT and Indian complete specification patent application titled “PRESERVATIVE-FREE ALLERGEN BLOCKER EYE GEL”, PCT application number: PCT/IB2024/053126 filed on 30 Mar. 2024 and Indian Application No. 202331024459 filed on 31 Mar. 2023. The entire collective teachings thereof being herein incorporated by reference.

### TECHNICAL FIELD

The present disclosure is in the technical field of novel in situ gel formulation for prevention of chronic eye allergies. More particularly, disclosure deals with a formulation which is preservative-free, isotonic with ocular environment and does not cause any irritation.

### BACKGROUND OF THE DISCLOSED EMBODIMENT

The ocular surface is directly and continuously exposed to external environment making it vulnerable to microbes, allergens and pollutants. According to NCBI data, eye allergies affect approximately 10% to 30% of the world population.

Around 98% of the affected population suffers either from seasonal allergic conjunctivitis or perennial allergic conjunctivitis. Its manifestation varies from seasonal allergic conjunctivitis, perennial allergic conjunctivitis to the chronic and potentially sight-threatening vernal keratoconjunctivitis (VKC), atopic keratoconjunctivitis and contact blepharoconjunctivitis.

Air pollution and climate change are potential drivers for the increasing burden of allergic ocular diseases. While 15-25% of the US population and 50% of the Europe population are affected by VKC, higher prevalence is seen in temperate zones of the Mediterranean areas, central Africa, Middle East, Japan, India, and South America.

VKC is a severe eye allergy which predominantly affects children between the age of 5-15 years in their school going years thus severely impact their quality of life. Patients experience symptoms such as watery, itchy, red, sore, swollen and stinging eyes on daily basis with exacerbations during peak allergic season. In India, VKC appeared to be more perennial with seasonal exacerbations and affecting higher age groups.

Unfortunately, in India most cases go undiagnosed, or patients opt for self-targeting leading to further blinding complications. Current therapies mostly use mast cell stabilizers and antihistamines to manage the mild symptoms. However, these therapies fail to manage severe cases and steroids are used for quick relief. Since steroids have severe side effects, they are not recommended for long-term management.

The current eye drops available in the market require multiple dosing and comes with preservatives which affect ocular epithelium integrity allowing entry to allergens/irritants causing subsequent inflammation and not completely effective in preventing the allergic response.

Therefore, there is a need to develop a preventive ophthalmic barrier that is isotonic and preservative-free, which will block allergens and help in management and prevention of chronic eye allergies.

### SUMMARY OF THE DISCLOSED EMBODIMENT

According to the aspect of the disclosure, an aqueous formulation for preventing eye allergies in a subject, the formulation comprises: at least three polymers, a pH regulator, and water, the at least three polymers are present in the formulation at a concentration ranging from 0.1% w/v to 2% w/v, the at least three polymers are selected from a group comprising tamarind seed polysaccharide, D-trehalose dihydrate, gellan gum, sodium hyaluronate, sodium carboxymethyl cellulose and poloxamer-407, a gel comprising the at least three polymers is formed in-situ at a physiological temperature upon instillation of the formulation onto ocular surface of the subject.

According to an exemplary aspect of the disclosure, an aqueous formulation as mentioned above, the at least three polymers comprise D-trehalose dihydrate at a concentration of 0.1-0.5% w/v, gellan gum at a concentration of 0.1-1% w/v, and sodium hyaluronate at a concentration of 0.1-0.5% w/v, an osmolarity measurement of the in-situ gel formulation prepared using the at least three polymers comprising the D-trehalose dihydrate, gellan gum, and sodium hyaluronate is in the range of 150-350 mOsm/kg.

According to another aspect of the disclosure, the aqueous formulation as mentioned above, the at least three polymers comprise tamarind seed polysaccharide at a concentration of 0.5-2% w/v, gellan gum at a concentration of 0.1-1% w/v, sodium hyaluronate at a concentration of 0.1-1% w/v, and D-trehalose dihydrate at a concentration of 0.1-0.5% w/v, the osmolarity measurement of the in-situ gel formulation prepared using the at least three polymers comprising the tamarind seed polysaccharide, gellan gum, sodium hyaluronate, and D-trehalose dihydrate is in the range of 150-350 mOsm/kg.

According to yet another aspect of the disclosure, an aqueous formulation as mentioned above, the at least three polymers comprise sodium carboxymethyl cellulose at a concentration of 0.1-1% w/v, gellan gum at a concentration of 0.1-1% w/v, and poloxamer-407 at a concentration of 0.01-0.5% w/v, the osmolarity measurement of the in-situ gel formulation prepared using the at least three polymers comprising the sodium carboxymethyl cellulose, gellan gum, and poloxamer-407 is in the range of 150-350 mOsm/kg.

According to an exemplary aspect of the disclosure, an aqueous formulation as mentioned above, the tamarind seed polysaccharide is obtained as a solution.

According to a further aspect of the disclosure, an aqueous formulation as mentioned above, the gel over the ocular surface is formed by a reaction with tears on the ocular surface within two minutes.

According to further aspect of the disclosure, an aqueous formulation as mentioned above, the pH regulator is tromethamine.

According to yet another aspect of the disclosure, an aqueous formulation as mentioned above, the formulation has a pH value in the range of 4.0 to 7.0.

According to further aspect of the disclosure, an aqueous formulation as mentioned above, the subject is a human being or an animal.

According to an exemplary aspect of the disclosure, a method of preparing an aqueous formulation for preventing eye allergies, the method comprises: (i) mixing at least three polymers selected from a group comprising tamarind seed polysaccharide, D-trehalose dihydrate, gellan gum, sodium hyaluronate, sodium carboxymethyl cellulose and poloxamer-407 at a concentration ranging from 0.1% w/v to 2% w/v in double distilled water at a temperature between 20° C. to 25° C. using a magnetic stirrer from 300 RPM to 500 RPM to obtain a clear solution, the clear solution is adjusted to a pH of about 7 with a pH regulator; (ii) the clear solution was filtered through a nylon syringe filter of size 0.22 μm by application of pressure manually to obtain the aqueous formulation; and (iii) transferring the aqueous formulation to a sterile eye dropper bottle aseptically and storing at 25° C. in a cool and dry place.

According to further aspect of the disclosure, an aqueous formulation as mentioned above, the tamarind seed polysaccharide is obtained as a solution, the tamarind seed polysaccharide solution is prepared by (i) solubilizing the tamarind seed polysaccharide in warm water by stirring for about 1 to 2 minutes, (ii) filtering the tamarind seed polysaccharide solution through a filter paper to obtain the tamarind seed polysaccharide solution.

According yet another aspect of the disclosure, an aqueous formulation as mentioned above, the pH regulator is tromethamine.

According to an exemplary aspect of the disclosure, an aqueous formulation as mentioned above, the filter paper is a 125 mm diameter Whatman no. 1 strip.

Several aspects of the disclosed embodiment are described below with reference to examples for illustration. However, one skilled in the relevant art will recognize that the disclosed embodiment can be practiced without one or more of the specific details or with other methods, components, materials and so forth. In other instances, well-known structures, materials, or operations are not shown in detail to avoid obscuring the features of the disclosed embodiment. Furthermore, the features/aspects described can be practiced in various combinations, though only some of the combinations are described herein for conciseness.

### BRIEF DESCRIPTION OF THE DRAWINGS

Example embodiments of the present disclosure will be described with reference to the accompanying drawings briefly described below.

FIG. 1 illustrates the epithelial barrier dysfunction in ocular allergies, according to the aspects of the disclosed embodiment.

FIG. 2 illustrates FTIR study of polymers, and formulation 1. FTIR spectrum of (a) trehalose, (b) sodium hyaluronate, (c) gellan gum, (d) mannitol and (e) formulation 1 scanned with diamond ATR spectrophotometer from 600 cm⁻¹ to 4000 cm⁻¹ to check the compatibility, according to the aspects of the disclosed embodiment.

FIG. 3 illustrates FTIR study of polymers, and formulation 2. FTIR spectrum of (a) tamarind seed polysaccharide, (b) D-trehalose dihydrate, (c) gellan gum, (d) sodium hyaluronate, (e) mannitol and (f) formulation 2 scanned with diamond ATR spectrophotometer from 600 cm⁻¹ to 4000 cm⁻¹ to check the compatibility, according to the aspects of the disclosed embodiment.

FIG. 4 illustrates FTIR study of polymers, and formulation 3. FTIR spectrum of (a) gellan gum, (b) sodium carboxymethyl cellulose, (c) poloxamer-407, (d) mannitol and (e) formulation 3 scanned with diamond ATR spectrophotometer from 600 cm⁻¹ to 4000 cm⁻¹ to check the compatibility, according to the aspects of the disclosed embodiment.

FIG. 5 illustrates clarity and gel formation study of formulation 1 at (a) laboratory (without STF) and (b) physiological condition (with STF), according to the aspects of the disclosed embodiment.

FIG. 6 illustrates clarity and gel formation study of formulation 2 at (a) laboratory (without STF) and (b) physiological condition (with STF), according to the aspects of the disclosed embodiment.

FIG. 7 illustrates clarity and gel formation study of formulation 3 at (a) laboratory (without STF) and (b) physiological condition (with STF), according to the aspects of the disclosed embodiment.

FIG. 8 illustrates the % light transmittance of distilled water and F1, according to the aspects of the disclosed embodiment.

FIG. 9 illustrates the % light transmittance of distilled water and F2, according to the aspects of the disclosed embodiment.

FIG. 10 illustrates the % light transmittance of distilled water and F3, according to the aspects of the disclosed embodiment.

FIG. 11 illustrates morphology of RBCs. Isotonicity study performed by haemolytic method and observed under an optical microscope at 100×10 magnifications under fluorescence microscope, according to the aspects of the disclosed embodiment.

FIG. 12 illustrates morphology of RBCs. Isotonicity study performed by haemolytic method and observed under an optical microscope at 100×10 magnifications under fluorescence microscope, according to the aspects of the disclosed embodiment.

FIG. 13 illustrates morphology of RBCs. Isotonicity study performed by haemolytic method and observed under an optical microscope at 100×10 magnifications under fluorescence microscope, according to the aspects of the disclosed embodiment.

FIG. 14 illustrates photographs of sterility study after 14 days for F1, according to the aspects of the disclosed embodiment.

FIG. 15 illustrates photographs of sterility study after 14 days for F2, according to the aspects of the disclosed embodiment.

FIG. 16 illustrates photographs of sterility study after 14 days for F3, according to the aspects of the disclosed embodiment.

FIG. 17 illustrates percent transmittance of F1 in sterility study, according to the aspects of the disclosed embodiment.

FIG. 18 illustrates percent transmittance of F2 in sterility stud, according to the aspects of the disclosed embodiment.

FIG. 19 illustrates percent transmittance of F3 in sterility study, according to the aspects of the disclosed embodiment.

FIG. 20 illustrates flow behavior of formulation 1, according to the aspects of the disclosed embodiment.

FIG. 21 illustrates flow behavior of formulation 2, according to the aspects of the disclosed embodiment.

FIG. 22 illustrates flow behavior of formulation 3, according to the aspects of the disclosed embodiment.

FIG. 23 illustrates mechanical and impedance study of F1, according to the aspects of the disclosed embodiment.

FIG. 24 illustrates mechanical and impedance study of F2, according to the aspects of the disclosed embodiment.

FIG. 25 illustrates mechanical and impedance study of F3, according to the aspects of the disclosed embodiment.

FIG. 26 illustrates no. of scratches in mice at 10 minutes, according to the aspects of the disclosed embodiment.

FIG. 27 illustrates no. of scratches in mice at 30 minutes, according to the aspects of the disclosed embodiment.

FIG. 28 illustrates no. of scratches in mice at 1 hour, according to the aspects of the disclosed embodiment.

FIG. 29 illustrates ocular irritation study with Formulation 1, according to the aspects of the disclosed embodiment.

FIG. 30 illustrates ocular irritation study with Formulation 3, according to the aspects of the disclosed embodiment.

FIG. 31 illustrates ocular irritation study with Formulation 3, according to the aspects of the disclosed embodiment.

In the drawings, like reference numbers generally indicate identical, functionally similar, and/or structurally similar elements. The drawing in which an element first appears is indicated by the leftmost digit(s) in the corresponding reference number.

### DETAILED DESCRIPTION OF THE DISCLOSURE

It is to be understood that the present disclosure is not limited in its application to the details of construction and the arrangement of components set forth in the following description or illustrated in the drawings. The present disclosure is capable of other embodiments and of being practiced or of being carried out in various ways. Also, it is to be understood that the phraseology and terminology used herein is for the purpose of description and should not be regarded as limiting.

The use of “including”, “comprising” or “having” and variations thereof herein is meant to encompass the items listed thereafter and equivalents thereof as well as additional items. The terms “a” and “an” herein do not denote a limitation of quantity, but rather denote the presence of at least one of the referenced items. Further, the use of terms “first”, “second”, and “third”, and the like, herein do not denote any order, quantity, or importance, but rather are used to distinguish one element from another. Further, the terms “comprising,” “including,” “having,” and the like, as used with respect to embodiments of the present disclosure, are synonymous. As used herein, the terms “agent,” “component,” or “ingredient” are used interchangeably and refer to a particular item that includes one or more chemical compounds (e.g., a food item from one or more plants that comprise one or more naturally occurring chemical compounds).

The term “component” or “ingredient” as used herein, refers to a particular item that includes one or more chemical compounds, i.e., edible compounds sourced from plants, fungi or algae that comprise one or more naturally occurring chemical compounds.

Alternative embodiments of the present disclosure and their equivalents may be devised without parting from the spirit or scope of the present disclosure. It should be noted that any discussion herein regarding “one embodiment”, “an embodiment”, “an exemplary embodiment”, and the like indicate that the embodiment described may include a particular feature, structure, or characteristic and that such particular feature, structure, or characteristic may not necessarily be included in every embodiment. In addition, references to the foregoing do not necessarily comprise a reference to the same embodiment. Finally, irrespective of whether it is explicitly described, one of ordinary skill in the art would readily appreciate that each of the particular features, structures, or characteristics of the given embodiments may be utilized in connection or combination with those of any other embodiment discussed herein.

As used herein, the singular forms “a”, “an”, and “the” include both singular and plural referents unless the context clearly dictates otherwise. By way of example, “a dosage” refers to one or more than one dosage.

The terms “comprising”, “comprises” and “comprised of” as used herein are synonymous with “including”, “includes” or “containing”, “contains”, and are inclusive or open-ended and do not exclude additional, non-recited members, elements or method steps.

All documents cited in the present specification are hereby incorporated by reference in their totality. In particular, the teachings of all documents herein specifically referred to are incorporated by reference.

Example embodiments of the disclosed embodiments are described with reference to the accompanying figures.

In the drawings, like reference numbers generally indicate identical, functionally similar, and/or structurally similar elements. The drawing in which an element first appears is indicated by the leftmost digit(s) in the corresponding reference number.

### Definitions

The term “allergic conjunctivitis” refers to an inflammatory reaction of the conjunctiva due to allergy.

The term “eye gel” refers to a gel formulation used for prevention of chronic eye allergies.

The term “tear” refers to the fluid secreted by the lacrimal glands that moistens and protects the ocular surface.

The term “preservative-free” refers to a product that does not contain any synthetic or natural preservatives.

The term “safety profile” refers to the history of a product's safety in use, based on clinical studies and other data.

The term “visual light transmission” refers to the ability of light to pass through a material without being absorbed or scattered.

The term “isotonic” refers to having the same concentration of solutes as the ocular environment.

The term “foreign bodies” refers to objects entering the eye causing irritation, infection, or damage.

The term “epithelial integrity” refers to the ability of the epithelial cells to maintain their structure and function.

The term “components” refers to the ingredients or parts that make up a product.

The term “allergens” refers to substances that cause an allergic reaction in individuals who are sensitive to them.

The term “pollutants” refers to harmful substances or particles that can cause damage to the ocular surface or other tissues.

The term “management” refers to the process of treating or controlling a condition or disease.

The term “physical protection” refers to the ability of a product to physically block or shield against external foreign bodies or other harmful substances.

The term “barrier concept” refer to the idea of creating a physical barrier between the eye and potential allergens or other harmful substances.

The term “multiple applications” refer to the potential for a product to be used for various purposes or conditions.

The term “ocular disorder” refers to any condition or disease affecting the eye or ocular tissues.

The term “pH regulator” refers to the acidity regulators, or pH control agents, are food additives used to change or maintain pH (acidity or basicity). They can be organic or mineral acids, bases, neutralizing agents, or buffering agents.

The term “Tamarind seed polysaccharide (TSP)” refers to a biocompatible, non-ionic polymer with antioxidant properties.

The term “osmolarity” refers to the number of particles of solute per liter of solution, whereas the term osmolality refers to the number of particles of solute per kilogram of solvent.

The term “Gellan gum” refers to an anionic polysaccharide polymer with sol-to-gel transformation characteristics in the presence of cations, and thus is used as an electrolyte-sensitive gelling agent.

### I) Embodiments of the Disclosure

An aqueous formulation for preventing eye allergies in a subject, the formulation comprises: at least three polymers, a pH regulator, and water, the at least three polymers are present in the formulation at a concentration ranging from 0.1% w/v to 2% w/v, the at least three polymers are selected from a group comprising tamarind seed polysaccharide, D-trehalose dihydrate, gellan gum, sodium hyaluronate, sodium carboxymethyl cellulose and poloxamer-407, a gel comprising at least three polymers (temperature and ion induced) is formed upon instillation of the formulation onto ocular surface of the subject.

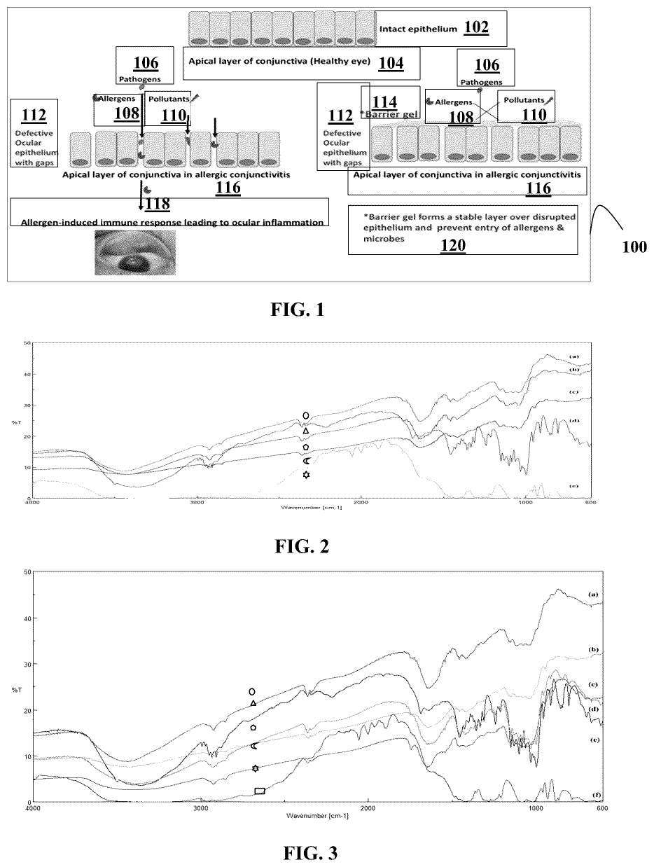

FIG. 1 (100) shows the epithelial (102) barrier dysfunction in ocular allergies in the apical layer of conjunctiva (healthy eye) (104). The flowchart consists of two main parts, left panel explains how pathogen (106), allergen (108), and pollutant (110) affect the eyes, leading to allergen-induced immune responses and ocular inflammation in allergic conjunctivitis. In an embodiment, the layer also consists of defective ocular epithelium with gaps (112). In addition, left panel shows that pathogens, allergens, and pollutants enter the eye and interact with the apical layer of conjunctiva (116), which is the outermost layer of the eye. These external stimuli can cause damage to the epithelial barrier, which is a protective layer that prevents the entry of harmful substances into the eye. The disrupted epithelium allows the allergens and other harmful substances to penetrate deeper into the eye, they can interact with immune cells and trigger an allergic response. This results in ocular inflammation, redness, itching, and other symptoms of allergic conjunctivitis (118). On right panel, it is depicted how the use of a barrier eye gel (114) can prevent conjunctivitis and restore the epithelial barrier. The barrier eye gel forms a stable layer over the disrupted epithelium, preventing the entry of allergens and microbes into the eye (120). In an embodiment, the barrier eye gel helps to reduce the severity of ocular inflammation and other symptoms of allergic conjunctivitis.

### II) Method of Preparation

A method of preparing an aqueous formulation for preventing eye allergies, the method comprises: (i) mixing at least three polymers selected from a group comprising tamarind seed polysaccharide, D-trehalose dihydrate, gellan gum, sodium hyaluronate, sodium carboxymethyl cellulose and poloxamer-407 in double distilled water at a temperature between 20° C. to 25° C. using a magnetic stirrer from 300 RPM to 500 RPM to obtain a clear solution, the clear solution is adjusted to a pH of about 7 with a pH regulator; (ii) the clear solution was filtered through a nylon syringe filter of size 0.22 μm by application of pressure manually to obtain the aqueous formulation; and (iii) transferring the aqueous formulation to a sterile eye dropper bottle aseptically and storing at 25° C. in a cool and dry place.

The disclosure has developed a novel in situ gel formulation for prevention of chronic eye allergies. The liquid formulation when instilled in the eye converts into a gel in the presence of tear, forming a protective layer over the ocular surface. The formulation is preservative free and does not contain any medicines/drug. It has ingredients with a well-established safety profile.

It is compatibility is demonstrated through its ability to not impair visual light transmission. Also, it is isotonic with ocular environment and does not cause any irritation. The components effectively block the entry of allergens into the eye, prevents loss of moisture and restore epithelial integrity. It can be used for both management and prevention of allergic conjunctivitis. It is basically an allergen blocker gel and so far, similar product with the barrier concept is not available in the market for ocular allergies. It has ability to physically protect against external foreign bodies, and that may lead to multiple applications related to ocular disorder.

Present disclosure has developed three different preservative-free barrier gel formulation for prevention of ocular allergies. Different combination of US FDA approved and biocompatible ophthalmic polymers were used to develop the barrier gel. The methodologies followed for detailed lab characterization and safety and efficacy studies are described as follows.

### Preliminary Screening

Suitable polymers for ophthalmic formulations were screened based on their ability to form clear (no undissolved particles) and transparent solutions (>85% transmittance) in water. Gellan gum, xanthan gum, carrageenan, arabinogalactose, sodium hyaluronate, D-trehalose dihydrate, tamarind seed polysaccharide, sodium carboxymethyl cellulose, poloxamer-407, hypromellose 940 and 980, were tested. Out of which, gellan gum, sodium hyaluronate, sodium carboxymethyl cellulose, poloxamer-407, tamarind seed polysaccharide, and D-trehalose dihydrate were found to form clear and transparent solutions in water and selected to develop differently in situ gel formulations. Different combinations of the polymer were used, keeping in view their FDA-approval limit or prior art. Different concentration of polymers was used for their optimum ability to form the gel. Subsequently, each combination was tested for its clarity, transparency, and gelling ability in the physiological condition.

The experimental design to optimize the in situ ophthalmic barrier formulation was carried out using Box-Behnken Design (BBD) on Design-Expert software (Stat-Ease, Inc., Minneapolis, Minnesota, USA) version 13.0.5.0.

TABLE 1
Optimized composition of F1, F2 and F3 formulations

| | F1 (% | F2 (% | F3 (% |
|---|---|---|---|
| Polymers | w/v) | w/v) | w/v) |
| D-trehalose dihydrate | 0.1-0.5 | 0.1-0.5 | |
| Gelrite gellan gum | 0.1-1 | 0.1-1 | 0.1-1 |
| Sodium Hyaluronate | 0.1-0.5 | 0.1-1 | |
| TSP | | 0.5-2 | |
| Sodium carboxymethyl cellulose | | | 0.1-1 |
| Poloxamer 407 | | | 0.01-0.5 |
| D (−)-Mannitol | 1-10 | 1-10 | 1-10 |

### Preparation of F1 Ophthalmic Barrier Formulation

The designed formulations were prepared by addition of D-trehalose dihydrate, gellan gum, and sodium hyaluronate at different concentrations in double distilled water at room temperature (20-25° C.) using a magnetic stirrer with temperature probe (iSTIR HP320, Neuation Technologies Pvt. Ltd., India) at 300 rpm. The addition of compounds was in the order mentioned above after the complete dissolution of each compound. Once the clear solution was obtained, the pH was checked using digital pH meter (alpha-01, Electronics India, India) and adjusted (about pH 7) with tromethamine, if required. Then the clear solution was filtered through a nylon syringe filter (0.22 μm, J-Sil, India) by application of pressure manually, transferred to a sterile eye dropper bottle aseptically, and stored at ambient conditions (25° C., cool and dry place) till further evaluation.

### Preparation of F2 Ophthalmic Barrier Formulation

### Prepare a Clear, Transparent Solution of Tamarind Seed Polysaccharide (TSP)

TSP is soluble in warm water (50° C.), sparingly soluble in cold water, and insoluble in methanol, acetone, and ether. One part of TSP solubilizes in 10-30 parts of warm water. TSP (1-5 mg) was added to 5-25 mL of warm water and stirred with a glass rod for 1-2 min. The TSP solution was filtered through qualitative filter paper (125 mm diameter Whatman no. 1), and the filtrate was completely clear and transparent. The resulting TSP solution strength was in the range 1-15%. TSP serial dilution was prepared in the range of 0.1-20%.

Preparation of Barrier In Situ Gel Formulation with TSP.

The formulations were prepared by taking different concentrations (Table 5) of TSP solution, gellan gum, sodium hyaluronate, and fixed concentration (0.1-0.5% w/v) of D-trehalose dihydrate were added to a glass beaker containing double distilled water and stirred using a magnetic stirrer at 500 rpm, till complete dissolution. Once the clear solution was obtained, the pH was checked and adjusted (about pH 7) with tromethamine, if required. Then the clear solution was filtered through a nylon syringe filter by application of pressure manually, transferred to a sterile eye dropper bottle aseptically, and stored at ambient conditions (25° C., cool and dry place) till further evaluation.

### Preparation of F3 Ophthalmic Barrier Formulation

The formulations were prepared by adding sodium carboxymethyl cellulose, gellan gum, and poloxamer-407 to a glass beaker containing a measured volume of freshly prepared double distilled water. Then the beaker containing polymeric solution was subjected to stirring at 500 rpm on a magnetic stirrer till the clear solution was obtained. Once the clear solution was obtained, the pH was checked using digital pH meter and adjusted (about pH 7) with tromethamine, if required. Then the clear solution was filtered through a nylon syringe filter by application of pressure manually, transferred to a sterile eye dropper bottle aseptically, and stored at ambient conditions (25° C., cool and dry place) till further evaluation.

### Evaluation of In Situ Gel for the Experimental Design

### Viscosity

The viscosity of the formulations was measured using small volume sample cup (DG26, SC4-27) of rotating viscometer (ViscoQC 300 type R, Anton Paar, Austria). The 1:4 ratio of simulated tear fluid (STF) (composition: sodium chloride (0.670 g), sodium bicarbonate (0.200 g), calcium chloride (0.008 g), magnesium chloride (0.005 g), potassium chloride (0.138 g) and water (98.979 g) [6] and formulation were taken in the sample cup. The readings were noted at 20 rpm at 34° C. with and without STF (pH 7.4).

### Mucoadhesion Study

The rheological synergism method is used to study the formulations' mucoadhesion. Briefly, mucin-type II (5% w/v) [9,10] was dispersed in the STF (pH 7.4) and equilibrated overnight at 4° C. The viscosity of formulation-mucin mixture (4:1) (ηₜ) and a pure formulation (ηₚ) and pure mucin (ηₘ) were measured at the shear rate of 6.8 s⁻¹ using spindle SC4-27 of rotational viscometer at 34° C. The adhesion force between formulation and mucin (I) b) was determined by equation (1).

$${η}_{b} = {η}_{t} - ( {η}_{p} + {η}_{m} ) \qquad ( 1 )$$

Then the mucoadhesion force (F) was calculated from equation (2)

$$F = {η}_{b} \cdot σ \qquad ( 2 )$$

Where σ is the shear rate (6.8 s⁻¹)

### Drug Polymer Compatibility Studies

Drug and gel-forming polymer compatibility were determined using a diamond ATR spectrophotometer (FTIR 4600 LE, Jasco, Japan). First, the FTIR spectra of plain polymer (4-5 mg) and optimized formulations (F1-F3) were recorded separately. The samples were scanned from 600 cm⁻¹ to 4000 cm⁻¹.

### Clarity Test

The formulations were visually inspected following the official protocols (USP-2020). Briefly, the formulations in transparent glass test tubes were held against a black and white background under illumination (2000-3750 lux). Visual inspection was done by swirling the test tubes to inspect undissolved particles. The formulations were categorized as “clear” based on the lack of undissolved particles observed against both backgrounds.

### Transparency

The percentage light transmittance of the formulation was measured by a UV-Visible spectrophotometer (Genova nano-38917, Jenway, UK). The formulation was scanned from 400 to 800 nm. The transmittance of distilled water with reference to air was measured before using it as a control.

### Determination of pH

The pH of the developed formulations and a marketed (Refresh Liquigel™, Allergan India Pvt. Ltd., Karnataka, India) eye drop was determined by a digital pH meter at 25±1° C. The pH meter was previously calibrated with the standard buffer solutions of pH 4.0, 7.0, and 9.2. The tromethamine was used to adjust the pH to 7-7.5.

### Gelling Time and Capacity

The test tube inversion method was used to study the gel time and capacity of the prepared formulations [12]. About 40 μL (USP 2020) of the formulation was mixed with freshly prepared STF (10 μL, pH 7.4) in a glass test tube. The test tube was kept in a digital water bath (RS Scientific, West Bengal, India) at 34° C. (ocular surface temperature). The time taken for sol-gel transition (gelling time) and capacity was measured visually by tilting the test tube. The gelling capacity was graded as follows: no gelling:-; gel formation after few minutes (>2 min.): +; gel formation immediately (≤2 min.): ++; immediate but rigid gel formation at room temperature: +++.

### Osmolarity and Isotonicity Evaluation

The osmolarity of the optimized formulations and the marketed formulation (Refresh Liquigel™, Allergan India Pvt. Ltd., Karnataka, India) was determined by the freezing point depression method using an osmometer (Osmomat 3000, Gonotech, Berlin, Germany). The osmolarity was adjusted with 1-10% w/v of mannitol solution. Since the mean osmolarity of human tears is about 310mOsm/kg, the formulation was optimized to this range. Further, the isotonicity of the optimized formulations was studied by the hemolytic method and compared with different saline concentrations. Briefly, the formulation (10 μL) was mixed with freshly collected blood on a glass slide. The developed slide was observed under a fluorescence microscope (Lawrence & Mayo, Mumbai, India) at 100×10 magnifications for the morphology of red blood cells (RBC). It was compared with hypotonic (0.1-5% w/v), hypertonic (0.1-5% w/v), and isotonic (0.1-5% w/v) saline solutions.

### Sterility Study

The sterility test of optimized formulation was done by direct inoculation, which is official in Indian Pharmacopeia 2010. The 0.5 mL of the ophthalmic formulation was aseptically transferred by using a sterile micropipette to 10 mL each of fluid thioglycollate (30-35° C.) and soybean-casein digest medium (20-25° C.) containing Staphylococcus aureus (ATCC 6538) and Candida albicans (ATCC MYA-2876TM) respectively and incubated along with positive and negative controls. The formulation was observed for the presence or absence of turbidity and compared with positive (microorganism in media), and negative (only media) controls for 14 days. The relative turbidity was assessed from the percentage transmittance of visible light (400-800 nm).

### Determination of Flow Behaviour or Rheology

### Mechanical Properties and Impedance Analysis

Mechanical properties of the optimized formulations (F1-F3) were studied by texture analyzer (Stable Microsystems, TA-HD plus, UK). The following parameters were measured. The analysis was performed by taking a 1:4 ratio of STF and the formulations and gelling them in a cylindrical holder.

The viscoelastic properties of the formulations (F1-F3) were measured by stress relaxation test (parameters: test mode; compression, pre-test, test, post-test speed; 1, 0.5, 1 mm/s, hold time; 60 s, load 5 Kg). The maximum force (F₀) and residual force (F₆₀) at the end of the relaxation phase were noted from the stress relaxation plot. The percentage stress relaxation (% SR) was calculated using equation 3. The viscosity index value was measured by performing a backward extrusion test (parameters: test mode; compression, pre-test, test, post-test speeds; 1, 0.5, 1 mm/s respectively, load 5 Kg, button mode). The graph's negative peak area is generally considered a viscosity index. The cohesiveness (area under the positive peak), gel strength/hardness/firmness (positive peak force), and stickiness (negative peak force) of (F1-F3) were measured by spreadability test (parameters: conical probe, distance; 23 mm, button mode).

$$\% SR = ( \frac{{F}_{0} - {F}_{60}}{{F}_{0}} ) \times 100 \qquad ( 3 )$$

The electrical properties were measured using an electrical impedance analyzer (NI-ELVIS-II system discovery 2, National Instruments, USA). The stainless-steel electrode was inserted in the sample, and the data were recorded in the range of 1-10,00000 Hz (parameters: reference resistor-1 MΩ2, probe resistance-1.04e+06Ω, probe capacitance-4.3e-11F, compensation-open short, amplitude-1V, amplification-1×).

### Mucoadhesion

The goat cornea was used as the representative mucosal layer for the study. The local slaughterhouse collected the goat eyeball in cold saline. The cornea pieces were attached to the base of the mechanical tester (Stable Microsystems, TA-HD plus, UK) with the help of double-sided acrylate tape. The flat probe was lowered at a speed of 0.5 mm/s, and a force of 10 g was applied on the mucosal surface for 10s to promote adhesion between the formulation and the mucosal layer. The probe was then retracted back at the same speed. The force required to separate the formulation from the corneal mucosal surface was noted as mucoadhesive force (negative area of peak).

### Stability Studies

A short term accelerated stability study (ICH Topic Q1A (R2)) of the optimized formulation was performed for 1 months [24]. The samples were filled in borosilicate glass vials and autoclaved for 15 mins at 121° C. It was stored in a stability chamber (Hally Instruments, Mumbai, India) at 40±2° C. and 75±5% relative humidity. After 10, and 30 days the samples were examined for pH, transparency and viscosity (with and without STF).

FTIR study of polymers, and formulation 1. FTIR spectrum of (a) trehalose, (b) sodium hyaluronate, (c) gellan gum, (d) mannitol and (e) formulation 1 scanned with diamond ATR spectrophotometer from 600 cm⁻¹ to 4000 cm⁻¹ to check the compatibility (FIG. 2).

FTIR study of polymers, and formulation 2. FTIR spectrum of (a) tamarind seed polysaccharide, (b) D-trehalose dihydratae, (c) gellan gum, (d) sodium hyaluronate, (e) mannitol and (f) formulation 2 scanned with diamond ATR spectrophotometer from 600 cm-1 to 4000 cm⁻¹ to check the compatibility (FIG. 3).

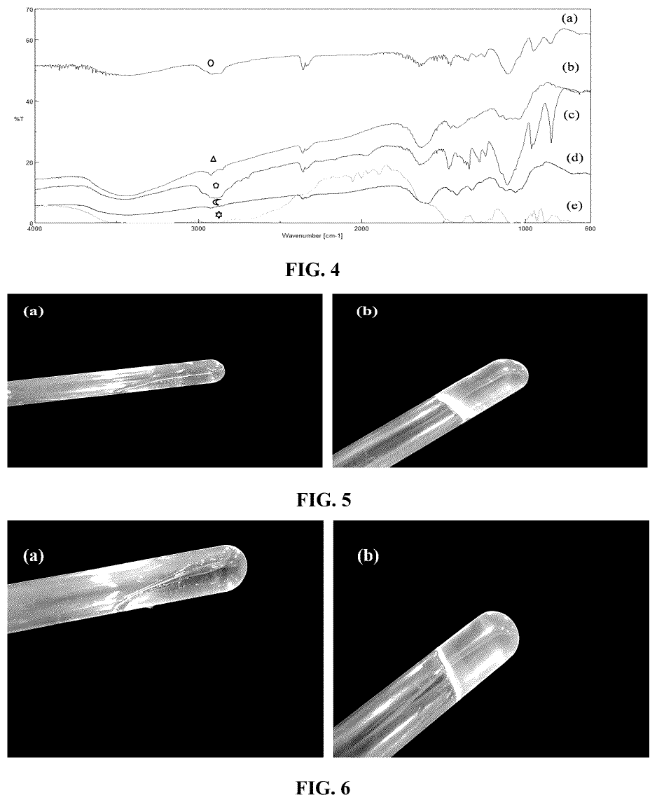

FTIR study of polymers, and formulation 3. FTIR spectrum of (a) gellan gum, (b) sodium carboxymethyl cellulose, (c) poloxamer-407, (d) mannitol and (e) formulation 3 scanned with diamond ATR spectrophotometer from 600 cm⁻¹ to 4000 cm⁻¹ to check the compatibility (FIG. 4).

### Results and Description

FTIR spectroscopy was used to evaluate the interaction between the polymers. The FTIR spectra of the individual polymers and the optimized formulations (F1-F3) are depicted in (FIGS. 2, 3, and 4). The characteristic peaks of D-trehalose dihydrate were observed at 3357.46 cm⁻¹ (O—H vibrations), 2933.20 cm⁻¹ (C—H stretching), 2907.16 cm⁻¹ (C—H asymmetric stretching), and 1686.44 cm⁻¹ (C═O stretching) (FIGS. 5a, and 6b) Sodium hyaluronate showed characteristic peaks at 2915.15 cm⁻¹ (C—H stretching), 1651.73 cm⁻¹ (carbonyl stretching) (FIGS. 2b, and 3d). The characteristic peaks of gellan gum were observed at 3433.64 cm⁻¹ (O—H stretching), 2923.56 cm⁻¹ (C—H stretching), and 1636.30 cm-1 (carbonyl stretching) (FIGS. 2c, 3c, and 4a). The characteristic peaks at 3397.96 cm⁻¹ (O—H stretching), 2924.52 cm⁻¹ (aliphatic C—H stretching), and 1646.91 cm⁻¹ (CH-OH stretching vibration) are assigned to the TSP (FIG. 3a). The sodium carboxymethylcellulose showed characteristic peaks at 3429.78 cm⁻¹ (—OH group stretching), 2361.41 cm⁻¹ (C—H stretching), 1600.41 cm⁻¹ (symmetric modes of stretching vibration of carboxylic groups), 1418.39 cm-1 (asymmetric modes of stretching vibration of carboxylic groups) (FIG. 7b). O—H stretching vibrations (3502.73 cm⁻¹), C—H stretching vibrations (2885.51 cm⁻¹), and C—O stretching vibrations (1111 cm⁻¹) are assigned to the poloxamer-407 (FIG. 4c).

### Clarity and Gel Formation Studies

FIG. 5 illustrates clarity and gel formation study of formulation 1 at (a) laboratory (without STF) and (b) physiological condition (with STF).

FIG. 6 illustrates clarity and gel formation study of formulation 2 at (a) laboratory (without STF) and (b) physiological condition (with STF).

FIG. 7 illustrates clarity and gel formation study of formulation 3 at (a) laboratory (without STF) and (b) physiological condition (with STF).

### Results and Description

Visual inspection of the prepared formulations (F1-F3) showed no undissolved particles. Thus, the preparations were clear (FIGS. 5, 6, and 7).

### Transmittance Studies

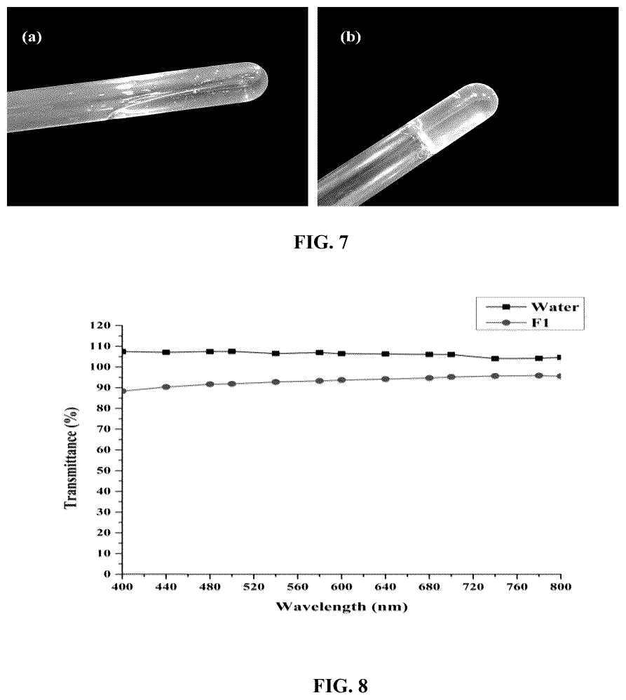

FIG. 8 illustrates the % light transmittance of distilled water and F1. The transmittance was measured by UV-Visible spectrophotometer in visible range. The data represent degree of transmittance of F1 as compared to water. Values represented as mean±S.D. (n=3).

FIG. 9 illustrates the % light transmittance of distilled water and F2. The transmittance was measured by UV-Visible spectrophotometer in visible range. The data represent degree of transmittance of F2 as compared to water. Values represented as mean±S.D. (n=3).

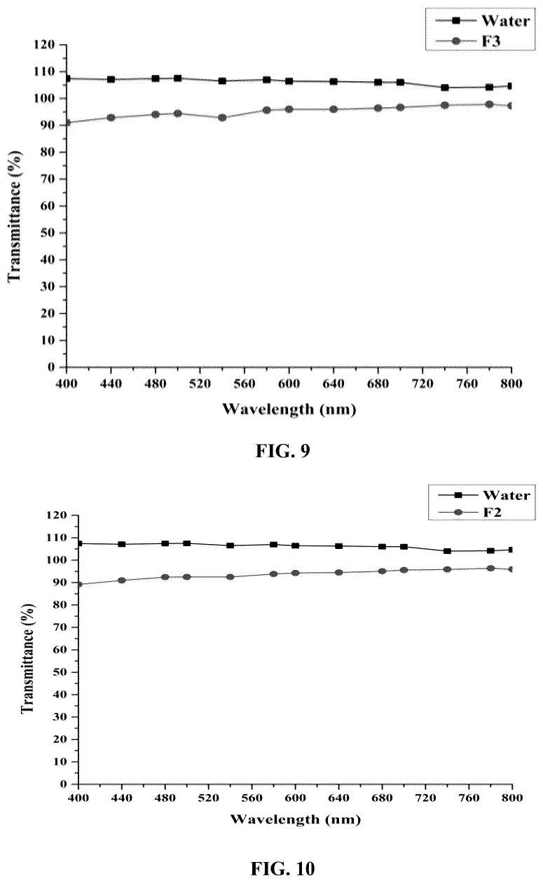

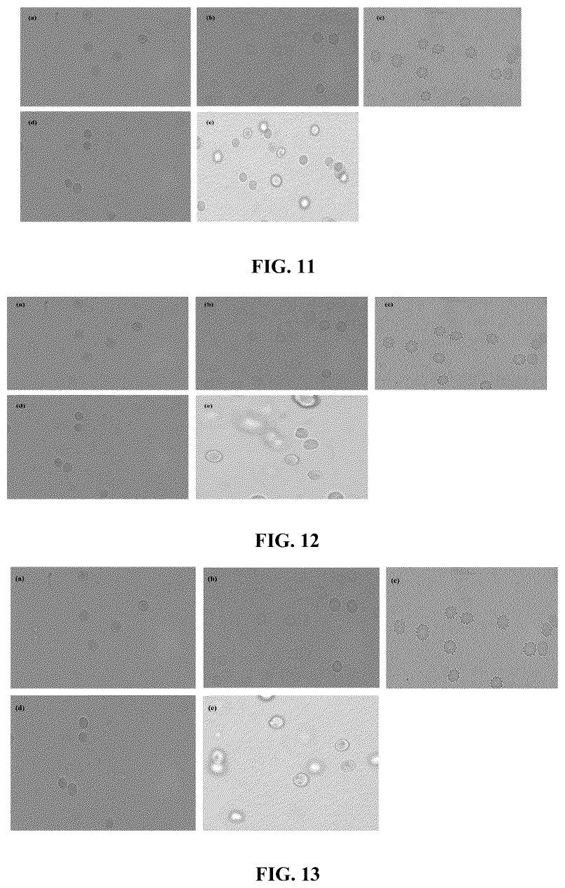

FIG. 10 illustrates the % light transmittance of distilled water and F3. The transmittance was measured by UV-Visible spectrophotometer in visible range. The data represent degree of transmittance of F3 as compared to water. Values represented as mean±S.D. (n=3).

### Results and Description

The transmittance of distilled water with reference to air was close to 100% in the visible range. The distilled water was used as a reference, and the percentage transmittance of the formulations (F1-F3) were found to be >87% in the visible range (400-800 nm) (FIGS. 8, 9, and 10). Since the light transmittance of vitreous is around 85-95%, the transparency of the formulations can be considered acceptable.

pH

The pH of the prepared formulations and marketed (Refresh Liquigel™, Allergan India Pvt. Ltd., Karnataka, India) eye drops were tested. The marketed eye drops showed a pH value 7.07. The pH of the optimized formulations (F1-F3) was 7.43+0.005, 6.57+0.05, and 7.23+0.005, respectively. A pH value of about 7.0 suggests their compatibility for application on the eye.

### Gelling Time and Capacity

The gelling capacity and time of optimized formulations were performed with STF by test tube tilting method. The three formulations (F1-F3) showed immediate gelation (++), within 35 sec.

FIG. 11 illustrates morphology of RBCs. Isotonicity study performed by hemolytic method and observed under an optical microscope at 100×10 magnifications under fluorescence microscope. Morphology of RBCs (a) without any formulation or saline solution, (b) hypotonic solution (c) hypertonic solution (d) isotonic solution and (e) F1.

FIG. 12 illustrates morphology of RBCs. Isotonicity study performed by hemolytic method and observed under an optical microscope at 100×10 magnifications under fluorescence microscope. Morphology of RBCs (a) without any formulation or saline solution, (b) hypotonic solution (c) hypertonic solution (d) isotonic solution and (e) F2.

FIG. 13 illustrates morphology of RBCs. Isotonicity study performed by hemolytic method and observed under an optical microscope at 100×10 magnifications under fluorescence microscope. Morphology of RBCs (a) without any formulation or saline solution, (b) hypotonic solution (c) hypertonic solution (d) isotonic solution and (e) F3.

### Results and Description

The osmolarity of the (F1-F3) was found to be in the range of 150-350 mOsm/kg. The marketed formulation (Refresh Liquigel™, Allergan India Pvt. Ltd., Karnataka, India) has an osmolarity of 276mOsm/kg. Since the acceptable range of osmolarity for ocular formulations is between 150 and 350mOsm/kg, the osmolarity of (F1-F3) were considered acceptable. Further, the hemolytic study was done to evaluate its isotonicity. Compared to the normal (FIG. 10a, 11a, and 12a), the hypotonic solution seems to have burster the RBCs (FIGS. 10b, 11b, and 12b). As expected, the hypertonic solution shrank them (FIGS. 10c, 11c, and 12c). The shape and size of RBCs remain unaltered when treated with isotonic solution (FIGS. 10d, 11d, and 12d) and formulations (F1-F3) (FIGS. 11e, 12e, and 13e).

### Sterility Studies

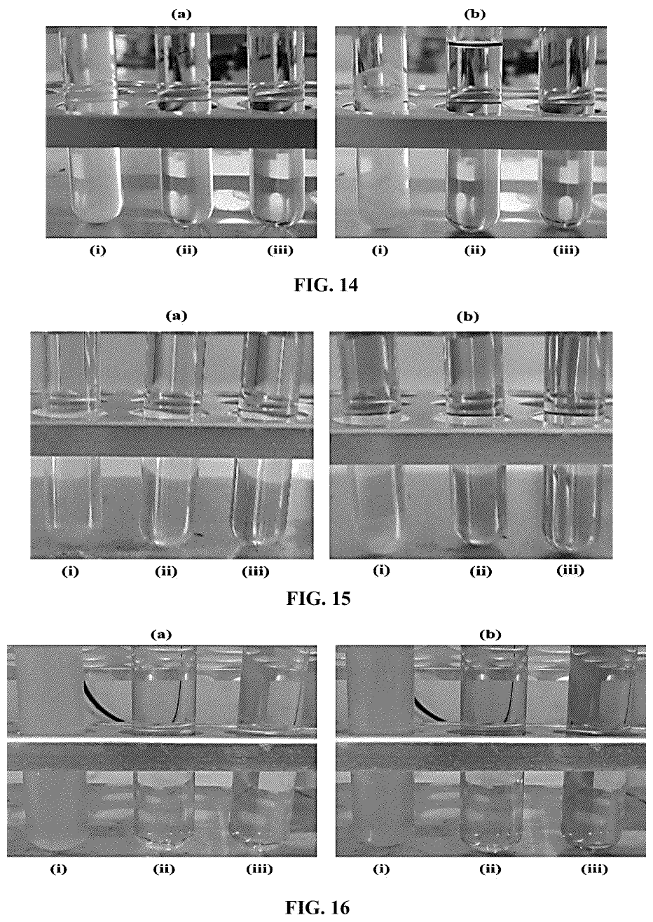

FIG. 14 illustrates photographs of sterility study after 14th days. (a) soyabean casein medium containing (i) +ve control, (ii) F1, (iii) −ve control; (b) fluid thioglycolate medium containing (i) +ve control, (ii) F1, (iii) −ve control.

FIG. 15 illustrates photographs of sterility study after 14th days. (a) soyabean casein medium containing (i) +ve control, (ii) F2, (iii) −ve control; (b) fluid thioglycolate medium containing (i) +ve control, (ii) F2, (iii) −ve control.

FIG. 16 illustrates photographs of sterility study after 14th days. (a) soyabean casein medium containing (i) +ve control, (ii) F3, (iii) −ve control; (b) fluid thioglycolate medium containing (i) +ve control, (ii) F3, (iii) −ve control.

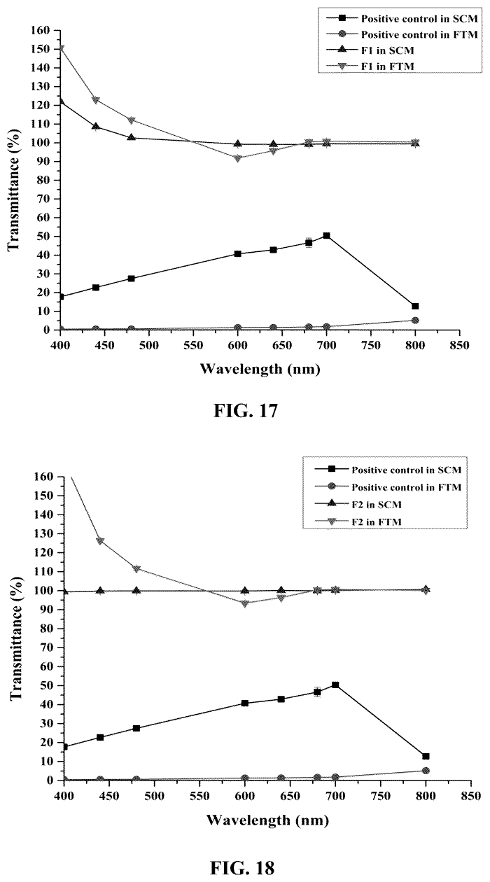

FIG. 17 illustrates percent transmittance of F1 in sterility study. The percent transmittance was measured by UV-Visible spectrophotometer in visible range for (a) F1 in fluid thioglycollate media, (b) F1 in soyabean casein media, (c) +ve control in soyabean casein media, (d) +ve control in fluid thioglycollate media after 14 days of the sterility study in incubator, fluid thioglycollate (30-35° C.) and soybean-casein digest medium (20-25° C.). Values represented as mean±S.D. (n=3).

FIG. 18 illustrates percent transmittance of F2 in sterility study. The percent transmittance was measured by UV-Visible spectrophotometer in visible range for (a) F2 in fluid thioglycollate media, (b) F2 in soyabean casein media, (c) +ve control in soyabean casein media, (d) +ve control in fluid thioglycollate media after 14 days of the sterility study in incubator, fluid thioglycollate (30-35° C.) and soybean-casein digest medium (20-25° C.). Values represented as mean±S.D. (n=3).

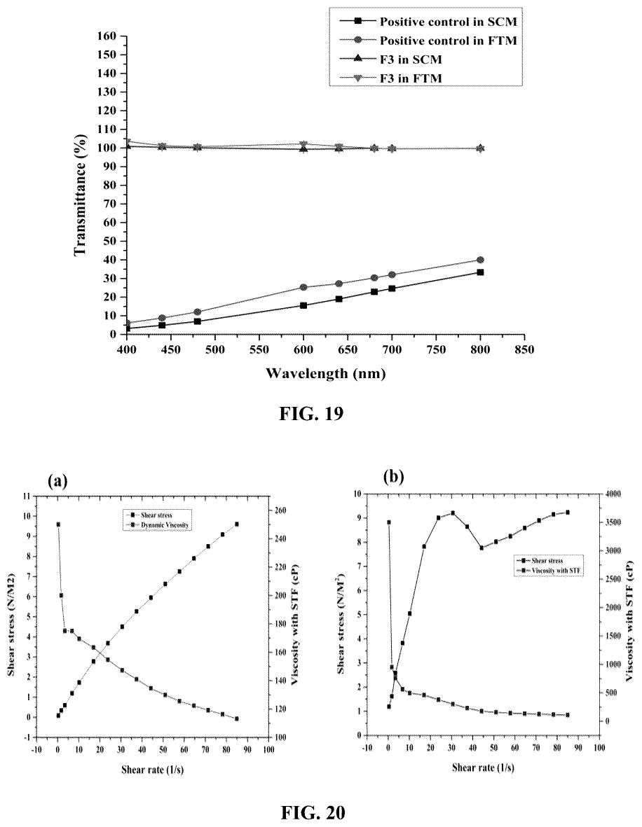

FIG. 19 illustrates percent transmittance of F3 in sterility study. The percent transmittance was measured by UV-Visible spectrophotometer in visible range for (a) F3 in fluid thioglycollate media, (b) F3 in soyabean casein media, (c) +ve control in soyabean casein media, (d) +ve control in fluid thioglycollate media after 14 days of the sterility study in incubator, fluid thioglycollate (30-35° C.) and soybean-casein digest medium (20-25° C.). Values represented as mean±S.D. (n=3).

### Results and Description

The sterility testing was done following the official methods (Indian Pharmacopoeia, 2010). Following visual observations (FIGS. 14, 15, and 16), the relative turbidity was plotted as percentage transmittance of visible light using the negative control (only media) as reference. As expected, the media containing microorganisms (positive control) showed turbidity (FIGS. 16, 17, and 18), which was also evident from their low transmittance. These results indicate the suitability of the media to promote the growth of microorganisms. Unlike the positive control, formulations (F1-F3) did not show any turbidity when present in both media.

### Flow Behaviour of Formulation Studies

(FIG. 20) illustrates the flow behaviour of formulation 1. Viscosity (cP) was measured as a function of rate of shear (s-1) using Anton Paar viscometer at 34° C. The data present decrease in viscosity of F1 with respect to change in shear rate. Values represented as mean±S.D. (n=3).

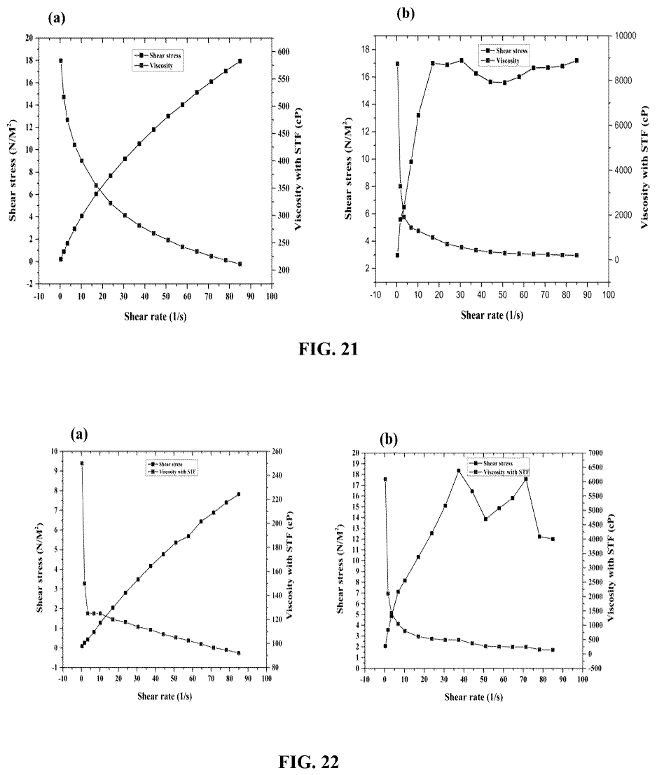

(FIG. 21) illustrates the flow behavior of formulation 2. Viscosity (cP) was measured as a function of rate of shear (s-1) using Anton Paar viscometer at 34° C. The data present decrease in viscosity of F2 with respect to change in shear rate. Values represented as mean±S.D.

(FIG. 22) illustrates the flow behavior of formulation 3. Viscosity (cP) was measured as a function of rate of shear (s-1) using Anton Paar viscometer at 34° C. The data present decrease in viscosity of F3 with respect to change in shear rate. Values represented as mean±S.D. (n=3)

### Results and Description

(FIGS. 19, 20, and 21) illustrates the rheological behavior of optimized formulation (F1-F3) respectively. All formulations (F1-F3) showed flow index (n value) (0.86502, 0.79998, 0.85755) respectively depicting newtonian flow due to its liquid state in non-physiological condition (without STF). In physiological condition (with STF at 34° C.) the flow index (n value) of (F1-F3) were (0.40631, 0.31236 and 0.37925) respectively which depict its pseudoplastic shear thinning behavior. With an increase in shear rate there is an increase in shear stress but with a dip in shear stress between shear rate of 30-50 l/sec. The decrease in viscosity with increase in shear rate observed is a desirable property.

### Mechanical and Impedance Studies

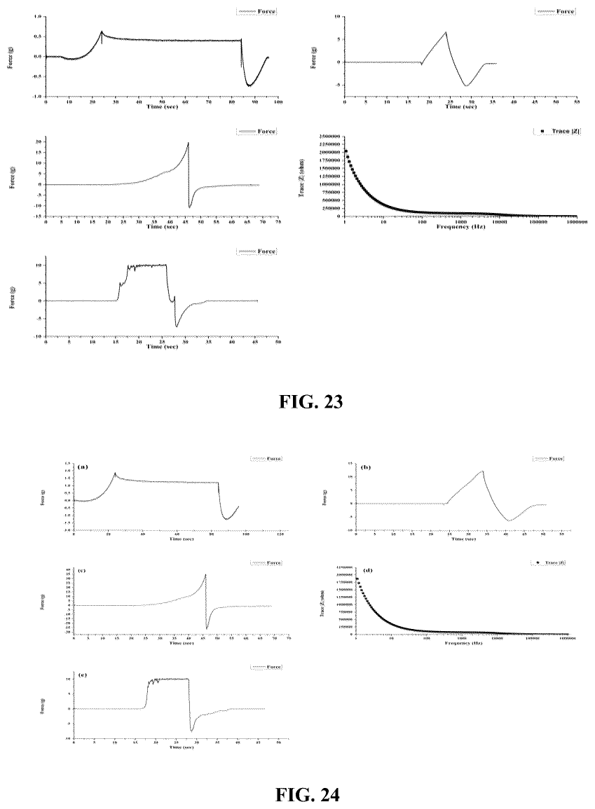

(FIG. 23) illustrates the mechanical and impedance study of F1. Mechanical properties (a) stress relaxation profile (b) backward extrusion (c) spreadability profile performed using texture analyzer; electrical property (d) impedance profile performed using electrical impedance analyzer. (e) mucoadhesion performed on goat cornea.

(FIG. 24) illustrates the mechanical and impedance study of F2. Mechanical properties (a) stress relaxation profile (b) backward extrusion (c) spreadability profile performed using texture analyzer; electrical property (d) impedance profile performed using electrical impedance analyzer. (e) mucoadhesion performed on goat cornea

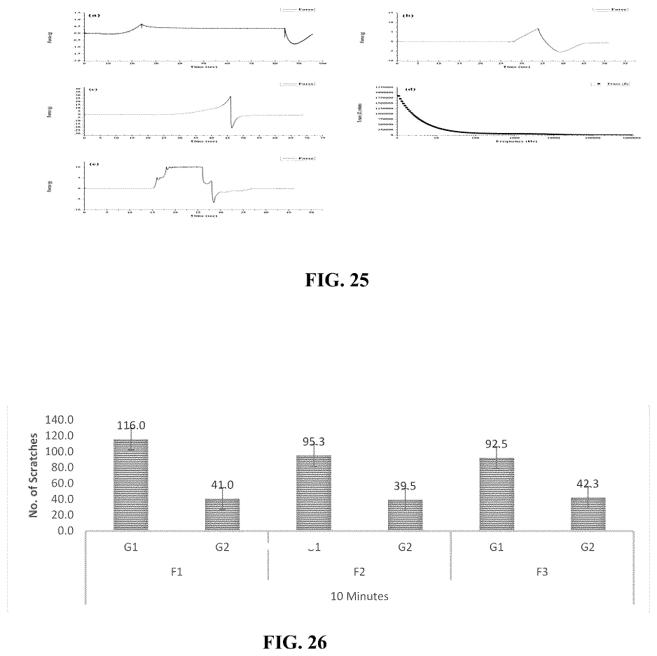

(FIG. 25) illustrates the mechanical and impedance study of F3. Mechanical properties (a) stress relaxation profile (b) backward extrusion (c) spreadability profile performed using texture analyzer; electrical property (d) impedance profile performed using electrical impedance analyser. (e) mucoadhesion performed on goat cornea.

### Results and Description

The texture profile analysis shows the force variation as a time function. The stress relaxation profile (FIGS. 23a, 24a, and 25a) of optimized (F1-F3) gel was analyzed to know the ability of the gel to absorb stress. The percentage shear relaxation of (F1-F3) were 33.3, 34.73, and 42.85%, respectively. It was reported that the percentage stress relaxation value between 37 and 42% suggests the viscoelastic nature of the formulation and has good mechanical strength. The backward extrusion test is a compression-extrusion test based on the resistance to flow (rheological property) through a small orifice, which the formulation experience when a force is applied. (F1-F3) viscosity, indexes were −22.942, −41.452, and −35.09 g·s, respectively (FIGS. 23b, 24b, and 25b) (viscosities with STF were 850, 983.5, and 1012.67 cP respectively). Spreadability refers to the ease with which a formulation can be spread. The spreadability (work of shear) is the inverse function of cohesiveness (area under the positive peak). The spreadability profile of (F1-F3) was represented in (FIGS. 23c, 24c, and 25c). Cohesiveness of (F1-F3) were −104.96, −179.921, and −140.93 g·s. The gel strength of ophthalmic in situ gel is a measure of the ability to develop and retain gel form. The gel strength and stickiness formulations were calculated from the spreadability profile in (FIGS. 23c, 24c, and 25c). The gel strength is the maximum positive force to deform the formulation and shows the formulation's strength. The higher firmness is better for the strength of the formulation. The positive peak force is related to the samples' firmness and provides information on the formulation's ease of spreadability. The firmness and stickiness of (F1-F3) were 19.79, 35.325, and 28.832 g and −10.891, −26.542, and −21.57 g, respectively. A similar type of result was reported in the literature. There are no pharmacopeial or regulatory standards for the mechanical properties of ophthalmic formulations. These texture analysis results revealed good enough gel strength to protect their structure on prolonged storage and ease of spreading, which is a favorable feature for topical ophthalmic administration.

The prepared (F1-F3) electrical properties were analyzed by recording the impedance profiles in the frequency range of 1-10,00000 Hz (FIGS. 23d, 24d, and 25d). The impedance profile of the pharmaceutical formulations is one of the critical tools for predicting the drug release profile from the formulations. The electrical impedance of the (F1-F3) decreased with an increase in frequency.

### Mucoadhesion

The negative area of the peak (FIGS. 24e, 25e, and 26e) is called adhesion. The mucoadhesive force of (F1-F3) were 15.481, −20.260, and −14.434 g·s. A similar type of result was reported in the literature. The results reveal that the formulations were mucoadhesive.

TABLE 2
Stability (30 days) data of formulations F1, F2, and F3.

| | | | Viscosity with |
|---|---|---|---|
| Day | pH | Viscosity (cP) | STF (cP) |
| Formulation-1 | | | |
| 0^{th} | 6.65 ± 0.006 | 133.47 ± 14.03 | 587.50 ± 12.5 |
| 10^{th} | 6.64 ± 0.006 | 188.13 ± 2.41 | 654.17 ± 19.09 |
| 30^{th} | 6.65 ± 0.03 | 87.47 ± 5.5 | 670.83 ± 14.43 |
| Formulation-2 | | | |
| 0^{th} | 6.92 ± 0.005 | 891.5 ± 19.15 | 1767 ± 6.92 |
| 10^{th} | 6.76 ± 0.005 | 1333.33 ± 28.87 | 2025.33 ± 69.32 |
| 30^{th} | 7.32 ± 0.005 | 131.33 ± 2.4 | 1283.3 ± 76.37 |
| Formulation-3 | | | |
| 0^{th} | 7.3 ± 0.005 | 183.07 ± 29.35 | 1012.7 ± 33.26 |
| 10^{th} | 6.82 ± 0.005 | 214 ± 3.27 | 908.33 ± 43.89 |
| 30^{th} | 6.39 ± 0.005 | 24.13 ± 5.44 | 345.67 ± 26.10 |

values represented as mean ±SD (n = 3).

### Results and Description

Stability (Table 2) study of optimized formulations (F1-F3) at the end of 1 month revealed that formulations were stable. No significant changes were observed in pH, viscosity.

### III) Ocular Irritation/Toxicity Study in Rabbits

The objective of ocular irritation studies (Draize technique) is to qualitatively and quantitatively assess ocular tolerance and irritability/toxicity potential of ophthalmic formulation upon administration to the eye. The objective of this study is to evaluate the possible irritation potential when a single dose of test item is instilled in the conjunctival sac of rabbit eye. This study will provide a rational basis for risk assessment in human.

### Protocol Followed

The GLP study was conducted at Edara Research Foundation (Hyderabad) after obtaining necessary ethical approval. A total 3 New Zealand rabbits (of either gender) weighing 1.5-3 kg was used for the study. The animals were quarantine and acclimatized to the laboratory environments for one week prior to the start of experiment with unrestricted access to both water and food. The animals were examined and weighed before the test and placed in specifically adapted cages, designed so as to avoid accidental injuries. They were observed for any signs of inflammation or visual abnormalities such as cataract or glaucoma. The rabbits were maintained in cages at 22-25° C. and fed at the same time every day till the day of the experiment. The test and reference formulations (100 μl) were administered in the conjunctival sac of the left eye of each animal after gently pulling the lower lid away from the eyeball. The lids are then gently held together for about 1 s in order to prevent loss of the material. The untreated right eye serves as a control. The eyes were monitored visually observed at 1, 2, 4, 12, 24, 48, and 72 h for any consequent inflammation or irritation reactions such as conjunctivitis, iritis, lacrimation, and redness. In vivo test is performed initially with one animal before proceeding to a confirmatory test in a second animal. If a corrosive or severe irritant effect is not observed in the initial test, the irritant or negative response will be confirmed using up to two additional animals.

### Result

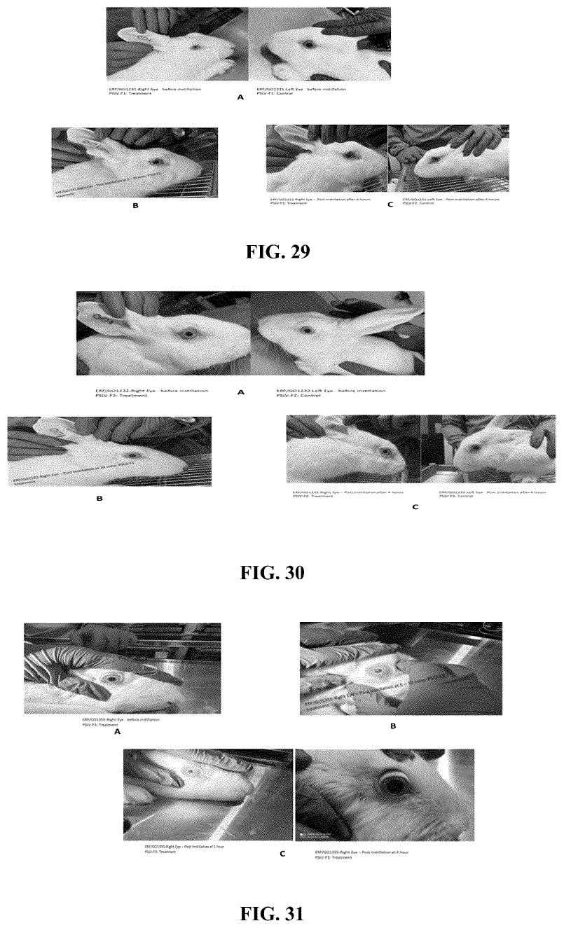

No ocular lesions were observed up to 48-hour post-test item instillation with all the three formulations. A. Right and left eye before formulation 1 (F1) instillation. Right eye is treated with formulation. Left eye is control. B. Right eye after 5 to 10 minutes of F1 instillation. C. Right and left eye after 4 hours of F1 instillation. Based on the outcome of the initial test (animal no. 001) (FIG. 29), confirmatory test was performed using two additional animals (animal no. 002 and 003) A. Right eye and left eye before formulation 2 (F2) instillation. Right eye is treated with formulation. Left eye is control. B. Right eye after 5 to 10 minutes of F2 instillation. C. Right and left eye after 4 hours of F2 instillation. (FIG. 30). A. Right before formulation 3 (F3) instillation. B. Right eye after 5 to 10 minutes of F3 instillation. C. Right eye after 1 and 4 hours of F3 instillation. (FIG. 31) and test item instillation procedure was performed in a similar manner of initial test. In both initial and confirmatory test (animal no. 001, 002 and 003), no ocular lesions were observed at 1, 24, 48, 72-hour post-test item instillation. Both treated and untreated eye of all the three rabbits were normal throughout the observation period.

No clinical signs of evident toxicity, moribundity and mortality were observed in any of the treated animals throughout the observation period. All the animals gained body weight over the course of study. The individual mean scores for animal no. 001, 002 and 003 at approximately 24, 48, 72-hour for cornea, iris, conjunctiva and chemosis were found to be 0. Results are shown in (FIGS. 2, 3 and 4).

### Conjunctival Allergen Challenge

### Test System Details:

Species: Mice
Strain: BALB/c
Sex: Male

Rationale for selection: Mice are commonly used species for efficacy studies.
No. of animals*: 13 (Pilot study-3; Main study: control-5 and treatment-5)
Age at treatment*: Between 8 to 12 weeks

Murine model of experimental allergic conjunctivitis (EAC) induced by SRW pollen was used for the allergen challenge study to check the efficacy of the formulations. The mouse EAC model was induced using previously reported methods with modifications.

In brief, animals were acclimatized to the experimental room conditions for a period of minimum three days prior to treatment. Animals were housed individually in polycarbonate cages (size: 290×210×140 mm as length, width and height respectively) with stainless steel top grill having facilities for holding feed and water. Animals were subjected to health assessment and allocated to G1 and G2 groups based on their body weight. Temperature of 22±3° C. and appropriate humidity of 30 to 70% was maintained.

| | | No. of animals |
|---|---|---|
| Group | Description | in each group |
| G1 | Control | 5 |
| | (Sensitization + Challenge + | |
| | Untreated) | |
| G2 | Treatment | 5 |
| | (Sensitization + Challenge + Treated) | |

Alum-Ragweed pollen (RW) Emulsion Preparation for sensitization of mice:

1 mg of RW was mixed with 1 ml of Imject™ alum and stirred over-night at room temperature to make Alum-RW emulsion.

### Protocol

Sensitization (Induction):

Day 0: For the first sensitization, 50 μL of the emulsified RW was injected into the left hind foot and root of the tail subcutaneously.

Day 14: For the second sensitization, 50 μL of the emulsified RW was injected into the right footpad subcutaneously.

### Challenge (RW Eye Drop Solution)

Days 26-29: An eye drop solution was prepared by mixing RW with PBS (2 mg in 10 μL/eye) and vortexed. RW-PBS eye drops will be mixed well before instillation into the eye. 10 μL of the RW eye drop solution will be instilled per eye from Days 26 to 29, once per day for 4 days.

After the instillation of the RW eye drops, the mouse was kept on a rough mesh on top of the mouse cage by gently holding the tail to keep the mouse from escaping. The mouse was allowed to blink and scratch for 3 min. This procedure will facilitate RW particle retention within the conjunctival sac and eyelids. 20 to 30 minutes after the last eye drop challenge, EAC will be evaluated using the 12-point scoring system based on the criteria described by Magone et. al.

### POC Evaluation

Effectiveness of barrier: The effectiveness of the barrier was checked by applying the gel and this gel treated animals were exposed to RW-PBS eye drops. After 20 to 30 minutes, eyes will be scored for inflammatory reactions. The control animals will remain untreated.

Duration of effectiveness: Post application of barrier (after gelling duration), RW-PBS eye drops were instilled at different time interval of barrier gel application (0-hour, 1, 2, and 4 hours.

Animals were examined clinically for signs of immediate hypersensitivity responses immediately after each topical challenge with RW pollen and other changes in body weight and moribundity and mortality check. The clinical grading, will be conducted by a trained personnel/veterinarian, using an ophthalmoscope or a dissecting microscope. The number of scratching behaviors will be counted as an indicator of the itching sensation (Table 3, 4 and 5).

TABLE 3
Representation of number of scratches in mice at
10 minutes after barrier gel application
At 10 Minutes

| | | No. of Scratches | | |
|---|---|---|---|---|
| Group | A. No. | F1 | F2 | F3 |
| G1 | 1 | 136 | 93 | 89 |
| | 2 | 108 | 98 | 97 |
| | 3 | 99 | 101 | 86 |
| | 4 | 121 | 89 | 98 |
| | Mean | 116.0 | 95.3 | 92.5 |
| G2 | 5 | 45 | 39 | 42 |
| | 6 | 38 | 52 | 46 |
| | 7 | 39 | 28 | 38 |
| | 8 | 42 | 39 | 43 |
| | Mean | 41.0 | 39.5 | 42.3 |

FIG. 26 illustrates no. of scratches in mice at 10 minutes.

TABLE 4
Representation of number of scratches in mice
at 30 minutes after barrier gel application
At 30 minutes

| | | No. of Scratches | | |
|---|---|---|---|---|
| Group | A. No. | F1 | F2 | F3 |
| G1 | 1 | 69 | 69 | 74 |
| | 2 | 62 | 71 | 68 |
| | 3 | 68 | 64 | 62 |
| | 4 | 79 | 59 | 65 |
| | Mean | 69.5 | 65.8 | 67.3 |
| G2 | 5 | 15 | 17 | 19 |
| | 6 | 24 | 20 | 18 |
| | 7 | 16 | 28 | 17 |
| | 8 | 14 | 12 | 14 |
| | Mean | 17.3 | 19.3 | 17.0 |

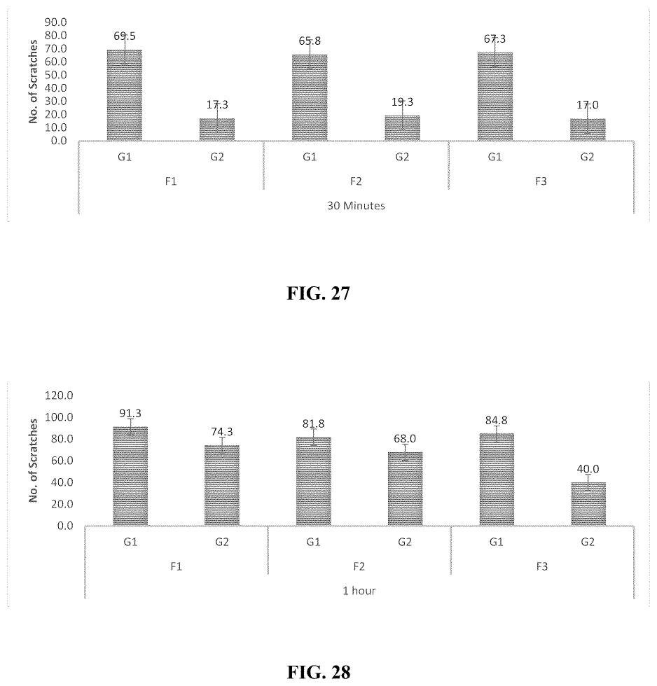

FIG. 27 illustrates no. of scratches in mice at 30 minutes.

TABLE 5
Representation of number of scratches in mice at
1 hour after barrier gel application
At 1^{st} hour

| | | No. of Scratches | | |
|---|---|---|---|---|
| Group | A. No. | F1 | F2 | F3 |
| G1 | 1 | 85 | 78 | 88 |
| | 2 | 98 | 84 | 92 |
| | 3 | 89 | 86 | 77 |
| | 4 | 93 | 79 | 82 |
| | Mean | 91.3 | 81.8 | 84.8 |
| G2 | 5 | 79 | 72 | 43 |
| | 6 | 71 | 69 | 49 |
| | 7 | 69 | 64 | 30 |
| | 8 | 78 | 67 | 38 |
| | Mean | 74.3 | 68.0 | 40.0 |

FIG. 28 illustrates no. of scratches in mice at 1 hour, according to the aspects of the disclosure.

TABLE 6
Table for the viscosity values obtained for the formulation.
The standard range of an ophthalmic formulation
should fall between 32.53 cP-1379 cP.

| | Specification | Observation |
|---|---|---|
| Formulation-1 | | |
| Ocular irritation | 32.53 cP-1379 cP | 300-500 cP |
| Allergen challenge | | 300-500 cP |
| Formulation-2 | | |
| Ocular irritation | | 200-500 cP |
| Allergen challenge | | 200-500 cP |
| Formulation-3 | | |
| Ocular irritation | | 100-500 cP |
| Allergen challenge | | 100-500 cP |

### IV) Uses, Applications and Benefits of the Disclosure

It is a novel in situ gel formulation with US FDA approved and biocompatible ophthalmic polymers, which forms a protective layer over ocular surface blocking the entry of allergens into the eye.

The barrier eye gel will help in prevention and management of chronic allergic conjunctivitis by blocking allergens entering the eye. The formulation components will also help in restoring damaged ocular epithelium and provide moisture similar to tear substitutes.

The barrier eye drop can also be used in dry eye disease and also therapeutic components can be added in formulation to treat the allergic conjunctivitis.

### V) Best Mode to Practice the

To prevent and manage chronic ocular allergies such as vernal keratoconjunctivitis.

Merely for illustration, only representative number/type of graph, chart, block, and sub-block diagrams were shown. Many environments often contain many more block and sub-block diagrams or systems and sub-systems, both in number and type, depending on the purpose for which the environment is designed.

While specific embodiments of the disclosed embodiments have been shown and described in detail to illustrate the inventive principles, it will be understood that the disclosed embodiments may be embodied otherwise without departing from such principles.

Reference throughout this specification to “one embodiment”, “an embodiment”, or similar language means that a particular feature, structure, or characteristic described in connection with the embodiment is included in at least one embodiment. Thus, appearances of the phrases “in one embodiment”, “in an embodiment” and similar language throughout this specification may, but do not necessarily, all refer to the same embodiment.

It should be understood that the figures and/or screen shots illustrated in the attachments highlighting the functionality and advantages of the disclosed embodiments are presented for example purposes only. The disclosed embodiments are sufficiently flexible and configurable, such that it may be utilized in ways other than that shown in the accompanying figures.

It should be understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. An aqueous formulation for preventing eye allergies in a subject, wherein the formulation comprises:
at least three polymers, a pH regulator, and water, wherein the at least three polymers are present in the formulation at a concentration ranging from 0.1% w/v to 2% w/v, wherein the at least three polymers are selected from a group comprising tamarind seed polysaccharide, D-trehalose dihydrate, gellan gum, sodium hyaluronate, sodium carboxymethyl cellulose and poloxamer-407, wherein a gel comprising the at least three polymers is formed in-situ at a physiological temperature upon instillation of the formulation onto ocular surface of the subject.

2. The aqueous formulation as claimed in claim 1, wherein the at least three polymers comprise D-trehalose dihydrate at a concentration of 0.1-0.5% w/v, gellan gum at a concentration of 0.1-1% w/v, and sodium hyaluronate at a concentration of 0.1-0.5% w/v, wherein an osmolarity measurement of the in-situ gel formulation prepared using the at least three polymers comprising the D-trehalose dihydrate, gellan gum, and sodium hyaluronate is in range of 150-350 mOsm/kg.

3. The aqueous formulation as claimed in claim 1, wherein the at least three polymers comprise tamarind seed polysaccharide at a concentration of 0.5-2% w/v, gellan gum at a concentration of 0.1-1% w/v, sodium hyaluronate at a concentration of 0.1-1% w/v, and D-trehalose dihydrate at a concentration of 0.1-0.5% w/v, wherein the osmolarity measurement of the in-situ gel formulation prepared using the at least three polymers comprising the tamarind seed polysaccharide, gellan gum, sodium hyaluronate, and D-trehalose dihydrate is in range of 150-350 mOsm/kg.

4. The aqueous formulation as claimed in claim 1, wherein the at least three polymers comprise sodium carboxymethyl cellulose at a concentration of 0.1-1% w/v, gellan gum at a concentration of 0.1-1% w/v, and poloxamer-407 at a concentration of 0.01-0.5% w/v, wherein the osmolarity measurement of the in-situ gel formulation prepared using the at least three polymers comprising the sodium carboxymethyl cellulose, gellan gum, and poloxamer-407 is in the range of 150-350 mOsm/kg.

5. The aqueous formulation as claimed in claim 1, wherein the tamarind seed polysaccharide is obtained as a solution.

6. The aqueous formulation as claimed in claim 1, wherein the gel over the ocular surface is formed by a reaction with tears on the ocular surface within two minutes.

7. The aqueous formulation as claimed in claim 1, wherein the pH regulator is tromethamine.

8. The aqueous formulation as claimed claim 1, wherein the formulation has a pH value in the range of 4.0 to 7.0.

9. The aqueous formulation as claimed in claim 1, wherein the subject is a human being or an animal.

10. A method of preparing an aqueous formulation for preventing eye allergies, wherein the method comprises:
(i) mixing at least three polymers at a concentration ranging from 0.1% w/v to 2% w/v in double distilled water at a temperature between 20° C. to 25° C. using a magnetic stirrer from 300 RPM to 500 RPM to obtain a clear solution, wherein the at least three polymers are selected from a group comprising tamarind seed polysaccharide, D-trehalose dihydrate, gellan gum, sodium hyaluronate, sodium carboxymethyl cellulose and poloxamer-407, wherein the clear solution is adjusted to a pH of about 7 with a pH regulator;
(ii) the clear solution was filtered through a nylon syringe filter of size 0.22 μm by application of pressure manually to obtain the aqueous formulation; and
(iii) transferring the aqueous formulation to a sterile eye dropper bottle aseptically and storing at 25° C. in a cool and dry place.

11. The method as claimed in claim 10, wherein the tamarind seed polysaccharide is obtained as a solution, wherein the tamarind seed polysaccharide solution is prepared by (i) solubilizing the tamarind seed polysaccharide in warm water by stirring for about 1 to 2 minutes, (ii) filtering the tamarind seed polysaccharide solution through a filter paper to obtain the tamarind seed polysaccharide solution.

12. The method as claimed in claim 10, wherein the pH regulator is tromethamine.

13. The method as claimed in claim 10, wherein the filter paper is a 125 mm diameter Whatman no. 1 strip.
